(19) United States
(12) Patent Application Publication
(10) Pub. No.: US 2023/0390358 A1
(43) Pub. Date: Dec. 7, 2023

(54) METHOD FOR ALLEVIATING DEPRESSION USING TNFAIP3-INTERACTING PROTEIN (TNIP) 1

(71) Applicant: Kaohsiung Chang Gung Memorial Hospital, Kaohsiung City (TW)

(72) Inventors: Yi-Yung Hung, Kaohsiung City (TW); Hong-Yo Kang, Kaohsiung City (TW); Ching-Yi Tsai, Kaohsiung City (TW); Ya-Ling Huang, Kaohsiung City (TW)

(21) Appl. No.: 18/112,554

(22) Filed: Feb. 22, 2023

(30) Foreign Application Priority Data
Jun. 2, 2022 (TW) 111120589

(51) Int. Cl. A61K 38/17; A61P 25/24

(52) U.S. Cl. CPC A61K 38/1709; A61P 25/24

(57) ABSTRACT

Disclosed herein is a method for alleviating depression, which includes administering to a subject in need thereof a composition containing TNFAIP3-interacting protein (TNIP) 1. The composition is administered to the CA3 region of the subject's hippocampus.

### CROSS-REFERENCE TO RELATED APPLICATION

This application claims priority of Taiwanese Invention Patent Application No. 111120589, filed on Jun. 2, 2022, the content of which is incorporated by reference in its entirety.

### SEQUENCE LISTING XML

A computer readable file containing a sequence listing is being electronically co-filed herewith via EFS-Web. The computer readable file, submitted under 37 CFR § 1.831(a) and having the file name “40Z5888.XML”, was created on Feb. 21, 2023 and has a size of 9,399 bytes. The content of the computer readable file is hereby incorporated by reference in its entirety.

### FIELD

The present disclosure relates to a method for alleviating depression using TNFAIP3-interacting protein (TNIP) 1.

### BACKGROUND

Depression is one of the most important health care problems in developed countries. The main symptoms of depression include excessively low mood, loss of interest in things, significant weight gain or loss, easy fatigue or loss of energy, and recurring negative thoughts.

Currently, drugs used clinically to alleviate depression include selective serotonin reuptake inhibitors (SSRIs), serotonin-norepinephrine reuptake inhibitors (SNRIs), norepinephrine-dopamine reuptake inhibitors (NDRIs), serotonin modulators, tricyclic antidepressants (TCAs), and monoamine oxidase inhibitors (MAOIs). However, these drugs might not be able to achieve the desired therapeutic effect, and might also cause severe side effects and drug resistance.

Certain commonly used medications, such as antihypertensive agents, gastrointestinal drugs, and anti-inflammatory drugs, might cause depression. For example, glucocorticoid, which is clinically used as an anti-inflammatory drug, might induce depression in rhesus macaque (Qin D. et al. (2019), Front Neurosci. 13:188).

It has been reported in Chiang T. I et al. (2021), Brain Behav. Immun. 95:454-461 that the expression level of TNFAIP3-interacting protein (TNIP) 2 was significantly increased in patients with major depressive disorder (MDD), while no significant difference was observed on the expression levels of TNIP1 and TNIP3 among such patients and healthy subjects. In addition, the expression level of TNIP2 was positively correlated with the severity of depression, so TNIP2 is expected to be useful as a therapeutic target for depression.

In spite of the aforesaid, there is still a need to develop an effective way for alleviating depression.

### SUMMARY

Therefore, an object of the present disclosure is to provide a method for alleviating depression, which can alleviate at least one of the drawbacks of the prior art, and which includes administering to a subject in need thereof a composition containing TNFAIP3-interacting protein (TNIP) 1. The composition is administered to the CA3 region of the subject's hippocampus.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the TNIP1 expression rate determined in each group of Example 1, infra, in which the symbol “*” represents p<0.05 (compared with the control group);

FIG. 2 shows the immobility time determined in each group of Example 1, infra, in which the symbol “*” represents p<0.05 (compared with the control group); and

FIG. 3 shows the sucrose preference percentage determined in each group of Example 2, infra, in which the symbol “*” represents p<0.05 (compared with the eGFP control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for alleviating depression, which includes administering to a subject in need thereof a composition containing TNFAIP3-interacting protein (TNIP) 1. The composition is administered to the CA3 region of the subject's hippocampus.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

According to the present disclosure, the TNIP1 suitable for use in this disclosure may be obtained as commercial products (such as enQuire BioReagents, Cat. No. QP13774 and Prospec, Cat. No. PRO-005), or may be prepared using techniques well-known to those skilled in the art. For example, the TNIP1 may be prepared using a commercially available synthesis kit or a solid phase peptide synthesis method.

In certain embodiments, the TNIP1 suitable for use in this disclosure may be prepared using genetic engineering techniques well-known to and commonly used by those skilled in the art (such as Sambrook J, Russell DW (2001) Molecular Cloning: a Laboratory Manual, 3rd ed. Cold Spring Harbor Laboratory Press, New York; and Current Protocols in Molecular Biology, Ausubel ed., John Wiley & Sons, Inc., New York (1997)). For example, according to recombinant DNA techniques known in the art, a recombinant vector containing a coding sequence of TNIP1 (SEQ ID NO: 1) derived from Mus musculus can be introduced into a selected host cell by transformation or transfection, followed by expression of the coding sequence of TNIP1, so as to obtain the TNIP1 (SEQ ID NO: 2) (NCBI reference sequence: NP_067302.2). In certain embodiments, the coding sequence of TNIP1 (SEQ ID NO: 1) may be delivered to the CA3 region of the subject's hippocampus by recombinant adeno-associated virus (rAAV), followed by expression of the coding sequence of TNIP1, so as to obtain the TNIP1 (SEQ ID NO: 2).

In addition, a coding sequence of TNIP1 (SEQ ID NO: 3) derived from Homo sapiens can be expressed according to the abovementioned recombinant DNA techniques, so as to obtain a TNIP1 having an amino acid sequence of SEQ ID NO: 4 (NCBI reference sequence: NP_001239314.1).

In certain embodiments, the TNIP1 may be produced in the CA3 region of the subject's hippocampus by administration of a molecule or drug that can induce TNIP1 expression. Examples of the molecule or drug may include, but are not limited to, serotonin-norepinephrine reuptake inhibitors (SNRIs) (such as duloxetine) and peroxisome proliferator-activated receptor-γ (PPAR-γ) agonists (such as pioglitazone).

According to the present disclosure, the depression may be selected from the group consisting of major depressive disorder (MDD), persistent depressive disorder (PDD), atypical depression, treatment-resistant depression (TRD), and combinations thereof.

According to the present disclosure, the composition may be prepared in the form of a pharmaceutical composition. The pharmaceutical composition may be formulated into a dosage form suitable for parenteral administration using technology well known to those skilled in the art. For parenteral administration, the pharmaceutical composition according to the present disclosure may be formulated into an injection, e.g., a sterile aqueous solution or a dispersion. In certain embodiments, the pharmaceutical composition may be administered via intracranial injection.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, and the like. The choice and amount of the aforesaid agents are within the expertise and routine skills of those skilled in the art.

The dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### Examples

### General Experimental Materials:

1. Recombinant Adeno-Associated Virus 9 (rAAV9) Particles

The rAAV9 particle carrying green fluorescent protein (GFP) gene (abbreviated as AAV9-eGFP), and the rAAV9 particle carrying a coding sequence of TNFAIP3-interacting protein (TNIP) 1 (SEQ ID NO: 1) and GFP gene (abbreviated as AAV9-mTNIP1) used in the following experiments were synthesized by CBT-LS-AAV Core, National Taiwan University (Taipei, Taiwan). The AAV9-eGFP can express GFP, and the AAV9-mTNIP1 can express GFP and TNIP1 (SEQ ID NO: 2) (NCBI reference sequence: NP_067302.2).

### 2. Experimental Mice

Male C57BL/6 mice (8 weeks old, with a body weight of approximately 20 to 25 g) used in the following experiments were purchased from BioLasco Taiwan Co., Ltd. All the experimental mice were housed in an animal room under the following laboratory conditions: an alternating 12-hour light and 12-hour dark cycle, a temperature maintained at 22° C. to 25° C., and a relative humidity maintained at 40% to 60%. Furthermore, water and feed were provided ad libitum for all the experimental mice. All experimental procedures involving the experimental mice were in compliance with the legal provision of the Institutional Animal Care and Use Committee of Chang Gung Memorial Hospital, Taiwan, and were carried out according to the Guide for the Care and Use of Laboratory Animals of National Institutes of Health (NIH).

### General Procedures:

### 1. Statistical Analysis

All the experiments described below were performed in triplicates. The experimental data of all the test groups are expressed as mean±standard deviation (SD), and were analyzed using Student's t-test, so as to evaluate the differences between the groups. Statistical significance is indicated by p<0.05.

### Example 1. Evaluation for the Effect of Overexpression of TNIP1 in the CA3 Region of Hippocampus on Alleviation of Depression

### Experimental Procedures:

A. Overexpression of TNIP1 Using rAAV9 Particles

The C57BL/6 mice were divided into two groups, including a control group (n=6) and an experimental group (n=7). The mice in each group were anesthetized using isoflurane. Next, the AAV9-mTNIP1 (5×10⁷ vg/0.5 μL) was randomly injected into or around the CA3 region of the hippocampus (stereotactic coordinates relative to bregma: anterior-posterior (AP): −2.2 mm; medio-lateral (ML): ±2.6 mm; and dorso-ventral (DV): −2.4 mm) of the respective mouse in the experimental group at a rate of 0.1 μL/min using a stereotaxic instrument (David Kopf). In addition, the AAV9-eGFP (5×10⁷ vg/0.5 μL) was randomly injected into or around the CA3 region of the hippocampus of the respective mouse in the control group in the same manner as described above.

### B. Forced Swimming Test (FST)

On day 28 after injection of the rAAV9 particle, the respective mouse in each group was subjected to analysis of depression-like behavior using forced swimming test according to procedures slightly modified from those described by Hung Y. Y. et al. (2019), Cells, 8:1021. Briefly, a glass beaker (diameter: 10 cm) was filled with water (temperature: 22° C. to 25° C.) to a depth of 13 cm, and the mouse placed into the glass beaker was unable to touch the bottom of the glass beaker, and thus cannot escape therefrom or rest therein. Each mouse was subjected to FST for a time period of 5 minutes, and the time spent immobile or floating (i.e., the immobility time) was recorded by visual inspection. The longer the immobility time, the more serious the depression-like behavior was.

### C. Measurement of TNIP1 Expression Level in Hippocampus

After completion of the FST on day 28 as described in section B of this example, the respective mouse was sacrificed, and the brain tissue was obtained from the respective mouse carcass. The brain tissue was subjected to a fixation treatment with a 4% paraformaldehyde solution (Sigma-Aldrich, Cat. No. 158127) at 4° C. for 24 hours. The fixed tissue sample was then embedded with optimal cutting temperature compound (Sakura Finetek, Cat. No. 4583), followed by slicing to obtain a tissue section having a thickness of 30 μm.

The tissue section was subjected to immunofluorescence staining using rabbit anti-TNIP1 antibody (Invitrogen, Cat. No. PAS-24503) as a primary antibody and goat anti-rabbit IgG antibody (Invitrogen, Cat. No. A11036) as a secondary antibody according to techniques well-known and customary to those skilled in the art.

Next, the resultant stained tissue section was observed and photographed using an optical microscope (TissueGnostics GmbH, TissueFAXS PLUS, Austria) at a magnification of 200×, followed by analysis using ImageJ Imaging Software (version 1.50i), so as to measure the TNIP1 expression levels in the hippocampal CA1 and CA3 regions.

The TNIP1 expression rate was calculated using the following

Equation(I):

A=B/C (I)

where A=TNIP1 expression rate

B=TNIP1 expression level in the hippocampal CA3 region

C=TNIP1 expression level in the hippocampal CA1 region

In the experimental group, the mice with a TNIP1 expression rate greater than 1 (i.e., overexpression of TNIP1 in the hippocampal CA3 region) were classified as the CA3+ group, while the mice with a TNIP1 expression rate equal to 1 (i.e., no TNIP1 overexpression in the hippocampal CA3 region) were classified as the CA3− group.

Thereafter, the TNIP1 expression rates and the immobility times determined in the CA3+ group, the CA3− group, and the control group were analyzed according to the method described in section 1 of “General Procedures”.

### Results:

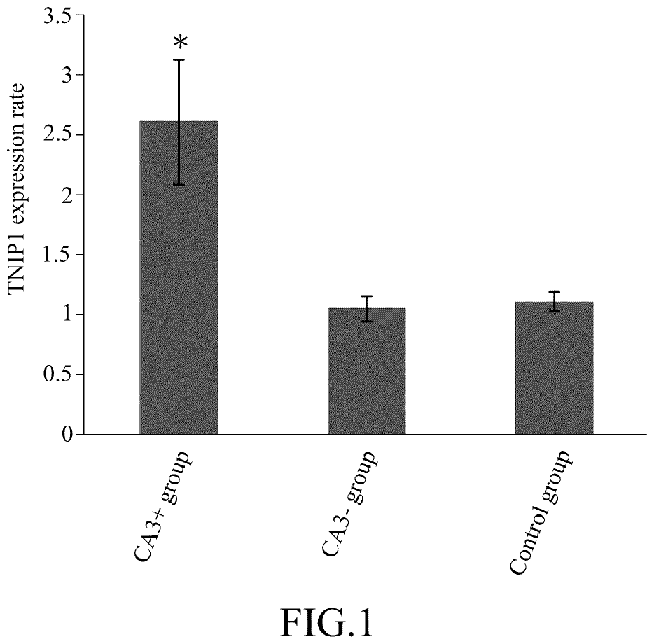

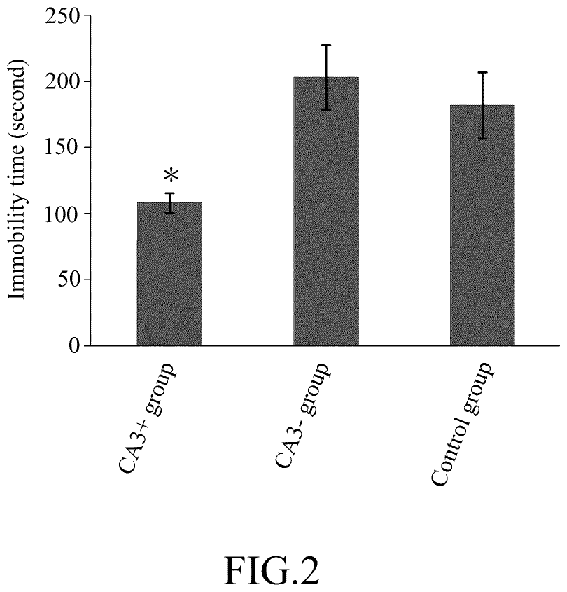

Referring to FIG. 1, the TNIP1 expression rate determined in the CA3+ group was significantly higher than those determined in the CA3− group and the control group. In addition, referring to FIG. 2, the immobility time determined in the CA3+ group was significantly shorter than those determined in the CA3− group and the control group.

These results indicate that overexpression of TNIP1 in the CA3 region of hippocampus using rAAV9 particles can exhibit an excellent effect in alleviating depression.

### Example 2. Evaluation for the Effect of Overexpression of TNIP1 in the CA3 Region of Hippocampus on Alleviation of Chronic Mild Stress-Induced Depression

### Experimental Procedures:

A. Overexpression of TNIP1 Using rAAV9 Particles

The C57BL/6 mice were divided into four groups, including an eGFP control group (n=11), a TNIP1 control group (n=9), a pathological control group (n=10), and an experimental group (n=10). Next, the AAV9-mTNIP1 (5×10⁷ vg/0.5 μL) was randomly injected into or around the CA3 region of the hippocampus of the respective mouse in the experimental group and the TNIP1 control group according to the procedures described in section A of Example 1. In addition, the AAV9-eGFP (5×10⁷ vg/0.5 μL) was randomly injected into or around the CA3 region of the hippocampus of the respective mouse in the eGFP control group and the pathological control group in the same manner as described in section A of Example 1.

### B. Sucrose Preference Test and Induction of Depression

On days 28, 42, 56, and 70 after injection of the rAAV9 particle, the mice in each group were subjected to sucrose preference test for 2 days according to procedures slightly modified from those described by Hung Y. Y. et al. (2019), supra. Briefly, the respective mouse was housed with one bottle of pure water from 9:00 am to 5:00 μm each day, and then the mouse was housed with one bottle of pure water and one bottle of 1% sucrose solution from 5:00 μm to 9:00 am the next day, and the total consumptions of pure water and sucrose solution during this period (i.e., a total period of 16 hours) were measured.

The sucrose preference percentage (%) was calculated using the following Equation (II):

D=[E/(E+F)]×100 (I)

where D=sucrose preference percentage (%)

E=total sucrose solution consumption (mL)

F=total pure water consumption (mL)

After completion of the sucrose preference test on day 28, the respective mouse in the experimental group and the pathological control group was subjected to induction of depression using a chronic mild stress (CMS) protocol slightly modified from that described by Schweizer M. C et al. (2009), PLoS One, 4(1):e4326. Briefly, various mild stressors were applied every day for a total period of 13 days. The mild stressors included white noise (90 dB); cage with 500 mL of water (water stress); removed bedding; soiled bedding; overnight illumination; space reduction in the cage (18 cm×8 cm×12 cm); food and water deprivation; tilted cages at 45 degrees; and repeated cold stress (10° C.).

### C. Measurement of TNIP1 Expression Level in Hippocampus

After completion of the sucrose preference test on day 70 as described in section B of this example, the respective mouse was sacrificed, and the brain tissue was obtained from the respective mouse carcass. The brain tissue was subjected to determination of TNIP1 expression rate according to the method described in section C of Example 1. Next, the mice with a TNIP1 expression rate greater than 1 (i.e., overexpression of TNIP1 in the hippocampal CA3 region) were selected from the TNIP1 control group and the experimental group, and the selected mice served as a selected TNIP1 control group and a selected experimental group, respectively.

Thereafter, the sucrose preference percentages determined in the eGFP control group, the pathological control group, the selected TNIP1 control group, and the selected experimental group were analyzed according to the method described in section 1 of “General Procedures”. The lower the sucrose preference percentage, the more serious the depression-like behavior was.

### Results:

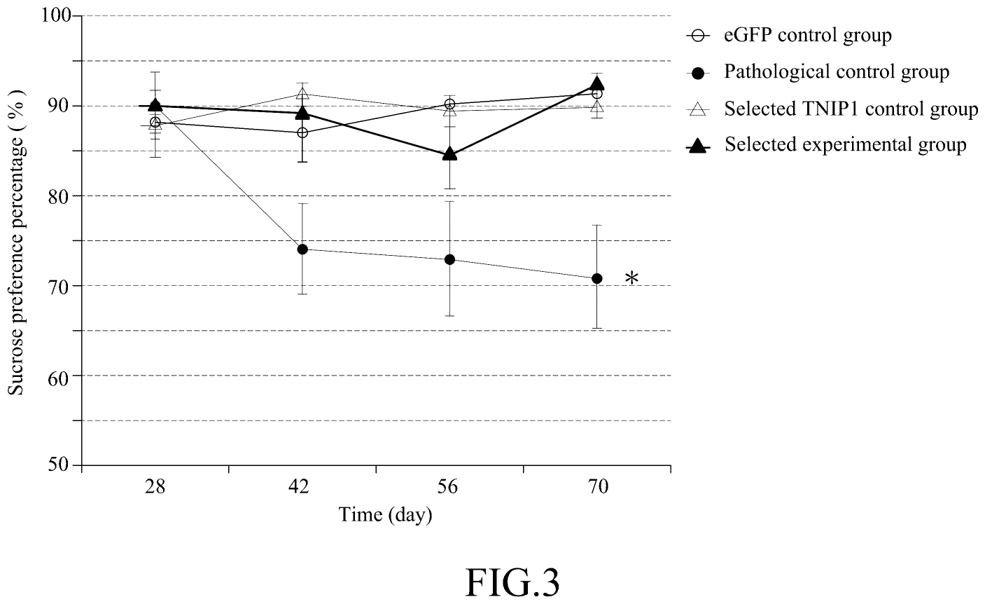

Referring to FIG. 3, in comparison with the eGFP control group, the sucrose preference percentage determined in the pathological control group decreased rapidly and significantly from day 28 to day 70, indicating that depression was successfully induced by chronic mild stress.

In addition, on days 42, 56, and 70, the sucrose preference percentage determined in the selected experimental group was significantly higher than that determined in the pathological control group. In particular, on days 42 and 70, the sucrose preference percentage determined in the selected experimental group was substantially similar to those determined in the eGFP control group and the selected TNIP1 control group.

These results indicate that the overexpression of TNIP1 in the CA3 region of hippocampus using rAAV9 particles can exhibit an excellent effect in alleviating depression.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating depression, comprising administering to a subject in need thereof a composition containing TNFAIP3-interacting protein (TNIP) 1,
wherein the composition is administered to the CA3 region of the subject's hippocampus.

2. The method as claimed in claim 1, wherein the composition is formulated as a pharmaceutical composition.

3. The method as claimed in claim 2, wherein the pharmaceutical composition is in a parenteral dosage form.

4. The method as claimed in claim 3, wherein the pharmaceutical composition is in a dosage form for intracranial injection.


SEQUENCE LISTING (4 sequences)

SEQ ID NO: 1 - DNA, 1944 bp, Mus musculus
  source 1..1944: mol_type = genomic DNA
atggaagggagaggaccctacgggatctacgacccagggggcagcacgcctctgggagaggtgtccgcagcttttgaacgtctagtggaggagaatactcggctgaagggaaaaatgcaagggataaagatgttaggggagcttctggaggagtctcagatggaagcgtccagactccggcagaaggcagaggagctggtcaaggacagcgagctgtcaccaccgacatctgccccctccttggtctcctttgatgacctggctgagctcacaggacaggatacaaaggtccaggtacatcctgctaccagcactgccgccaccaccaccgccaccgccaccacgggaaactccatggagaagcccgagccagcctccaaatctccgtccaatggcgcctcctcggactttgaagtggtccctactgaggagcagaattcacccgaaactggcagccaccctacgaacatgatggacctggggcccccacccccagaggacagcaacctgaagctccacctgcagcgcctggagaccacccttagcgtgtgtgcagaggagccagaccacagccagctcttcacccacctgggccgcatggccctcgagttcaacaggttggcctccaaagtgcataaaaatgagcagcgcacctccatcctgcagaccttatgtgagcagctgcgccaggagaatgaagccctgaaggccaagctggacaagggcctggaacagcgggatctggctgctgagaggctgcgggaggaaaacacggagctcaagaaactgttgatgaacagcagctgcaaagagggactctgtgggcagcccagctccccaaagccagagggtgctggcaagaagggcgtggctggacagcagcaggccagtgtgatggcgagtaaagtccctgaagcgggggcctttggagcagctgagaagaaggtgaagttgctagaacagcaacgcatggagctgctggaagtgaacaagcagtgggaccagcatttccggtccatgaagcagcagtatgagcagaagatcacagagcttcgccagaagctggtggacctgcagaaacaggtaactgagctggaggccgaacgggagcagaagcagcgtgactttgaccggaaactcctcctggccaaatcgaagatagagatggaagagaccgacaaggagcagctgacagcagaggccaaggaactgcgccagaaggtcaggtacctacaggatcagctgagcccgctcacaaggcaacgagaataccaggagaaggagatccagcggctcaataaggccctggaggaggccctcagcatccaggcctctccatcatctccgcctgcagcttttgggagtccagaaggcgttgggggccatctgaggaagcaggaactagtgacacagaatgagttgctgaaacagcaggtaaagatctttgaagaggacttccagagggaacggagtgaccgtgaacgcatgaatgaagagaaggaggagctgaagaagcaagtagagaagctgcaggcccaggtcaccctgactaatgcccagctcaaaactctcaaagaggaggagaaggccaaggaagccctcaaacagcagaagaggaaagcaaaggcttcgggagagcgctaccacatggaaccccaccctgagcacgtctgcggcgcctatccctatgcctacccacccatgccagccatggtacctcaccatgcctacaaggactggtcccagatccgataccctccaccccctgtgcccatggagcacccgcccccacaccccaactctcgcctcttccatctgccggagtacacctggcgtccaccctgtgcagggattcggaatcagagctctcaagtgatggacccgcccccagacaggcctgcagagccagagtctgcagacaatgactgtgatgggccccagtga

SEQ ID NO: 2 - protein, 647 aa, Mus musculus
MEGRGPYGIYDPGGSTPLGEVSAAFERLVEENTRLKGKMQGIKMLGELLEESQMEASRLRQKAEELVKDSELSPPTSAPSLVSFDDLAELTGQDTKVQVHPATSTAATTTATATTGNSMEKPEPASKSPSNGASSDFEVVPTEEQNSPETGSHPTNMMDLGPPPPEDSNLKLHLQRLETTLSVCAEEPDHSQLFTHLGRMALEFNRLASKVHKNEQRTSILQTLCEQLRQENEALKAKLDKGLEQRDLAAERLREENTELKKLLMNSSCKEGLCGQPSSPKPEGAGKKGVAGQQQASVMASKVPEAGAFGAAEKKVKLLEQQRMELLEVNKQWDQHFRSMKQQYEQKITELRQKLVDLQKQVTELEAEREQKQRDFDRKLLLAKSKIEMEETDKEQLTAEAKELRQKVRYLQDQLSPLTRQREYQEKEIQRLNKALEEALSIQASPSSPPAAFGSPEGVGGHLRKQELVTQNELLKQQVKIFEEDFQRERSDRERMNEEKEELKKQVEKLQAQVTLTNAQLKTLKEEEKAKEALKQQKRKAKASGERYHMEPHPEHVCGAYPYAYPPMPAMVPHHAYKDWSQIRYPPPPVPMEHPPPHPNSRLFHLPEYTWRPPCAGIRNQSSQVMDPPPDRPAEPESADNDCDGPQ

SEQ ID NO: 3 - DNA, 1860 bp, Homo sapiens
  source 1..1860: mol_type = genomic DNA
atggaagggagaggaccgtaccggatctacgaccctgggggcagcgtgccctcaggagaggcatccgcagcttttgagcgcctagtgaaggagaattcccggctgaaggaaaaaatgcaagggataaagatgttaggggagcttttggaagagtcccagatggaagcgaccaggctccggcagaaggcagaggagctagtgaaggacaacgagctgctcccaccaccttctccctccttgggctccttcgaccccctggctgagctcacaggaaaggactcaaatgtcacagcatctcccacagcccctgcatgccccagtgacaagccagcaccagtccagaagcctccatccagtggcacctcctctgaatttgaagtggtcactcctgaggagcagaattcaccagagagcagcagccatgccaatgcgatggcgctgggccccctgccccgtgaggacggcaacctgatgctgcacctgcagcgcctggagaccacgctgagtgtgtgtgccgaggagccggaccacggccagctcttcacccacctgggccgcatggccctggagttcaaccgactggcatccaaggtgcacaagaatgagcagcgcacctccattctgcagaccctgtgtgagcagcttcggaaggagaacgaggctctgaaggccaagttggataagggcctggaacagcgggatcaggctgccgagaggctgcgggaggaaaatttggagctcaagaagttgttgatgagcaatggcaacaaagagggtgcgtctgggcggccaggctcaccgaagatggaagggacaggcaagaaggcagtggctggacagcagcaggctagtgtgacggcaggtaaggtcccagaggtggtggccttgggcgcagccgagaagaaggtgaagatgctggagcagcagcgcagtgagctgctggaagtgaacaagcagtgggaccagcatttccggtccatgaagcagcagtatgagcagaagatcactgagctgcgtcagaagctggctgatttgcagaagcaggtgactgacctggaggccgagcgggagcagaagcagcgtgactttgaccgcaagctcctcctggccaagtccaagattgaaatggaggagaccgacaaggagcagctgacagcagaggccaaggagctgcgccaaaaggtcaagtacctgcaggatcagctgagcccactcacccgacagcgtgagtaccaggaaaaggagatccagcggctcaacaaggccctggaggaagcactgagcatccaaaccccgccatcatctccaccaacagcatttgggagcccagaaggagcaggggccctcctaaggaaacaggagctggtcacgcagaatgagttgctgaaacagcaggtgaagatcttcgaggaggacttccagagggagcgcagtgatcgtgagcgcatgaatgaggagaaggaagagctgaagaagcaagtggagaagctgcaggcccaggtcaccctgtcaaatgcccagctaaaagcattcaaagatgaggagaaggcaagagaagccctcagacagcagaagaggaaagcaaaggcctcaggagagcgttaccatgtggagccccacccagaacatctctgcggggcctacccctacgcctacccgcccatgccagccatggtgccacaccatggcttcgaggactggtcccagatccgctacccccctccccccatggccatggagcacccgcccccactccccaactcgcgcctcttccatctgcaccagttttgtcgcagccggaatacacctggcgtctaccctgtggaggggttcgaaatccaaatcagagctcccaagtga

SEQ ID NO: 4 - protein, 619 aa, Homo sapiens
MEGRGPYRIYDPGGSVPSGEASAAFERLVKENSRLKEKMQGIKMLGELLEESQMEATRLRQKAEELVKDNELLPPPSPSLGSFDPLAELTGKDSNVTASPTAPACPSDKPAPVQKPPSSGTSSEFEVVTPEEQNSPESSSHANAMALGPLPREDGNLMLHLQRLETTLSVCAEEPDHGQLFTHLGRMALEFNRLASKVHKNEQRTSILQTLCEQLRKENEALKAKLDKGLEQRDQAAERLREENLELKKLLMSNGNKEGASGRPGSPKMEGTGKKAVAGQQQASVTAGKVPEVVALGAAEKKVKMLEQQRSELLEVNKQWDQHFRSMKQQYEQKITELRQKLADLQKQVTDLEAEREQKQRDFDRKLLLAKSKIEMEETDKEQLTAEAKELRQKVKYLQDQLSPLTRQREYQEKEIQRLNKALEEALSIQTPPSSPPTAFGSPEGAGALLRKQELVTQNELLKQQVKIFEEDFQRERSDRERMNEEKEELKKQVEKLQAQVTLSNAQLKAFKDEEKAREALRQQKRKAKASGERYHVEPHPEHLCGAYPYAYPPMPAMVPHHGFEDWSQIRYPPPPMAMEHPPPLPNSRLFHLHQFCRSRNTPGVYPVEGFEIQIRAPK